(19) United States
(12) Patent Application Publication
(10) Pub. No.: US 2026/0207084 A1
(43) Pub. Date: Jul. 23, 2026

(54) AUDIOLOGICAL INTERVENTION

(71) Applicant: Cochlear Limited, Macquarie University, NSW (AU)

(72) Inventors: Birgit PHILIPS, Mechelen (BE); Bastiaan VAN DIJK, Rosmalen (NL)

(21) Appl. No.: 19/140,114

(22) PCT Filed: Dec. 21, 2023
(86) PCT No.: PCT/IB2023/063136

§ 371 Date: Jun. 17, 2025

Related U.S. Application Data
(60) Provisional application No. 63/477,283, filed on Dec. 27, 2022.

(51) Int. Cl. A61B 5/12; A61B 5/00

(52) U.S. Cl. CPC A61B 5/123; A61B 5/741; A61B 5/742

(57) ABSTRACT

Presented herein are techniques related to a method that includes: obtaining, at a processing device, results of a diagnostic test presented to a recipient of a hearing device; determining, from the results, that the recipient exhibits a random error or a non-random error with respect to an auditory stimulus presented in the diagnostic test; and selecting between a technological intervention associated with the hearing device or a rehabilitation intervention to be performed by the recipient based upon the determination that the recipient exhibits the random error or the non-random error.

### BACKGROUND

### Field of the Invention

The present invention relates generally to hearing devices.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In some aspects, the techniques described herein relate to a method, including: obtaining, at a processing device, results of a diagnostic test presented to a recipient of a hearing device; determining, from the results, that the recipient exhibits a random error or a non-random error with respect to an auditory stimulus presented in the diagnostic test; and selecting between a technological intervention associated with the hearing device or a rehabilitation intervention to be performed by the recipient based upon the determination that the recipient exhibits the random error or the non-random error.

In some aspects, the techniques described herein relate to a method including: administering an audiological test to a recipient of a hearing device, the administering including: presenting to the recipient via the hearing device, a plurality of speech sound auditory stimuli, presenting to the recipient via a user interface a plurality of responses for each of the plurality of speech sound auditory stimuli, and receiving from the recipient via the user interface, a response associated with each of the plurality of speech sound auditory stimuli; analyzing the responses associated with each of the plurality of speech sound auditory stimuli; and determining in response to the analyzing that the recipient exhibits a consistent error or an inconsistent error with respect to at least one of the plurality of speech sound auditory stimuli.

In some aspects, the techniques described herein relate to one or more non-transitory computer readable storage media including instructions that, when executed by a processor, cause the processor to: obtain results of a diagnostic test presented to a recipient of a hearing device; determine, from the results, that the recipient exhibits a random error or a non-random error with respect to an auditory stimulus presented in the diagnostic test; and select between a technological intervention associated with the hearing device or a rehabilitation intervention to be performed by the recipient based upon the determination that the recipient exhibits the random error or the non-random error.

In some aspects, the techniques described herein relate to a system, including: a processing device including a user interface and at least one processor, wherein the at least one processor is configured to: cause a hearing device to present a plurality of speech sound auditory stimuli to a recipient of the hearing device; present to the recipient via the user interface a plurality of responses for each of the plurality of speech sound auditory stimuli; receive from the recipient via the user interface a response associated with each of the plurality of speech sound auditory stimuli; analyze the responses associated with each of the plurality of speech sound auditory stimuli; and determine in response to the analyzing that the recipient exhibits a consistent error or an inconsistent error with respect to at least one of the plurality of speech sound auditory stimuli.

In some aspects, techniques described herein relate to a device, comprising: a memory; at least one processor, configured to initiate delivery of a plurality of speech sound auditory stimuli to a user of a hearing device; a user interface configured to display a plurality of responses for each of the plurality of speech sound auditory stimuli, and to receive a selection of one of the plurality of responses in association with each of the plurality of speech sound auditory stimuli; wherein the at least one processor is configured to analyze the responses associated with each of the plurality of speech sound auditory stimuli, and to determine in response to the analyzing that the user exhibits a consistent error or an inconsistent error with respect to at least one of the plurality of speech sound auditory stimuli.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a speech confusion matrix to which the phoneme error-based intervention techniques disclosed herein have been applied, according to an example embodiment;

FIG. 3 is a first mapping of phoneme errors to interventions, according to an example embodiment;

FIG. 4 is a second mapping of phoneme errors to interventions, according to an example embodiment;

FIG. 5 illustrates a speech confusion matrix and error identification for a first cochlear implant recipient, according to an example embodiment;

FIG. 6 illustrates a speech confusion matrix and error identification for a second cochlear implant recipient, according to an example embodiment;

FIG. 7 is a block diagram of a fitting system configured to implement the phoneme error-based intervention techniques disclosed herein, according to an example embodiment;

FIG. 8 is a flowchart illustrating a process flow for implementing the error identification aspects of the phoneme error-based intervention techniques disclosed herein, according to an example embodiment;

FIG. 9 is a flowchart illustrating a process flow for selecting an intervention according to the phoneme error-based intervention techniques disclosed herein, according to an example embodiment;

FIG. 10 is a flowchart illustrating a process flow for administering an audiological test according to the phoneme error-based intervention techniques disclosed herein, according to an example embodiment;

FIG. 11 is a schematic diagram illustrating an implantable stimulation system with which aspects of the techniques presented herein can be implemented; and

FIG. 12 is a schematic diagram illustrating a vestibular stimulator system with which aspects of the techniques presented herein can be implemented.

### DETAILED DESCRIPTION

Various factors are known to affect outcomes of hearing device users, particularly cochlear implant recipients who often struggle with a more severe level of hearing loss when compared to individuals who utilize other types of hearing devices. Some of these factors include duration of deafness, age at implantation, residual hearing, family involvement, and patient motivation. Even accounting for these differences, outcomes between cochlear implant recipients are widespread and variable, and even “good candidates” can present as “poor performers.” These varied outcomes, and particularly poor outcomes, may leave cochlear implant recipients and their communication partners dissatisfied and clinicians frustrated. Such poor outcomes also do little to encourage policy makers to increase resources and funding for cochlear implant surgery and rehabilitation.

Post-implantation outcomes are typically represented as a clinical speech perception score, in the form of a phoneme or word score in which the outcome is measured as the percent of the phonemes or words correctly identified by the subject of the test. While these outcome measures provide a high-level overview of a user's/recipient's outcomes, they do not provide more detailed information on the types of phoneme perception difficulties and errors that result in this score.

As a result, current aftercare (both fitting and auditory rehabilitation) is broad and does not focus on individual perception errors and perception error patterns recipients are making. Therefore, instead of targeted intervention, recipients are provided with a one-size-fits-all approach. The techniques disclosed herein provide a diagnostic test battery that unravels phoneme error patterns. Such tests can be used to diagnose the fine-grained phoneme errors for cochlear implant recipients and users/recipients of other hearing devices. The disclosed techniques also incorporate the phoneme error pattern test results into individualized aftercare pathways, which can ultimately improve speech perception outcomes.

Merely for ease of description, the techniques presented herein are primarily described with reference to a specific implantable medical device system, namely a cochlear implant system. However, it is to be appreciated that the techniques presented herein can also be partially or fully implemented by other types of hearing devices and implantable medical devices. For example, the techniques presented herein can be implemented by other hearing device (e.g., auditory prosthesis) systems that include one or more other types of hearing devices, such as hearing aids, middle ear auditory prostheses, bone conduction devices, direct acoustic stimulators, electro-acoustic prostheses, auditory brain stimulators, combinations or variations thereof, etc. The techniques presented herein can also be implemented by dedicated tinnitus therapy devices and tinnitus therapy device systems. The techniques presented herein can also be implemented in consumer hearing devices, such as Personal Sound Amplification Product (PSAP) devices, headphones, and earbuds, among others. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

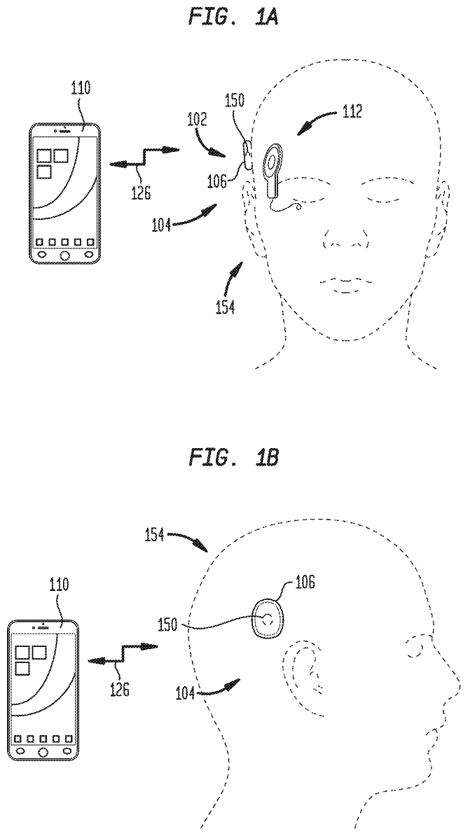

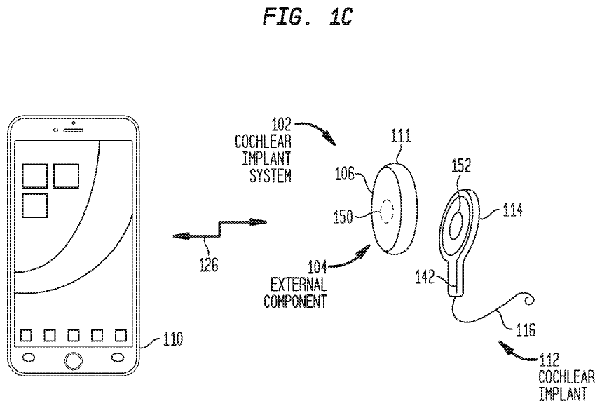

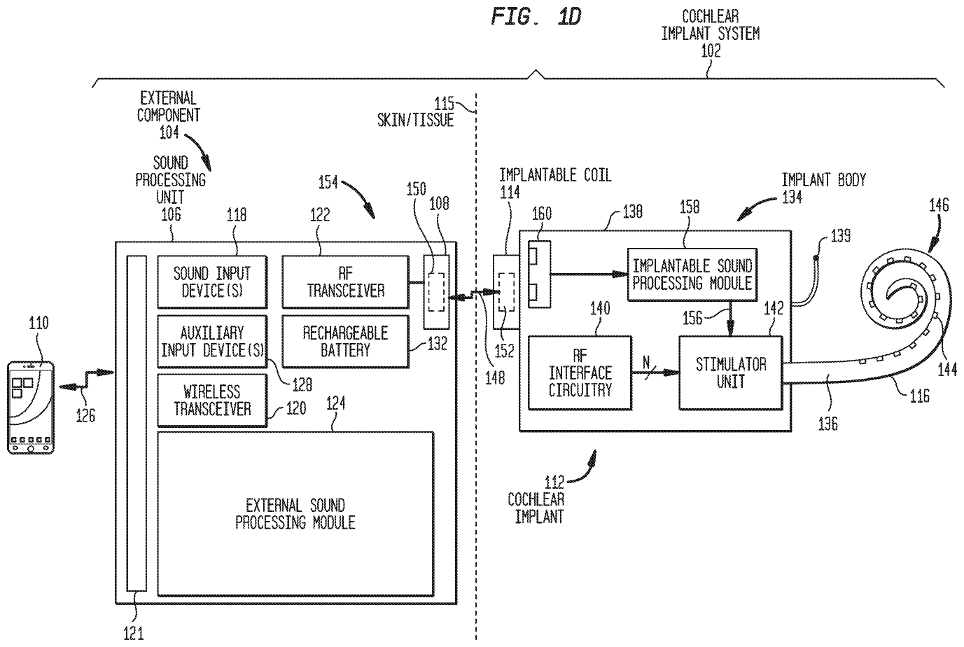

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component can comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. The external device 110 and the cochlear implant system 102 (e.g., OTE sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc. Accordingly, cochlear implant system 102 includes interface 121.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices can include additional types of input devices and/or less input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

As noted above, the techniques disclosed herein are directed to a diagnostic test battery unravelling auditory stimuli error patterns, such as phoneme or other speech sound error patterns, and to incorporating these test results in individualized aftercare pathways. As explained in more detail below, the techniques present hearing device users, such as hearing/auditory prosthesis recipients, with a diagnostic test battery that includes, for example, speech sounds such as phonemes or tonemes in the case of tonal languages. Based on results of the diagnostic tests, a type of intervention is selected. Certain test results will indicate that a technological intervention, such as cochlear implant fitting procedure, is likely to result in a better outcome. For instance, if the recipient does not discriminate two sounds, fitting parameters can be adapted to make the distinction between the two sounds larger. According to one example, if two phonemes are consistently confused by a recipient, the recipient's cochlear implant can be fit to focus on the spectral differences between the confused phonemes. Accordingly, the upper stimulation levels in the channels that represent the largest difference between the two phonemes can be increased, enhancing the contrast in the recipient's perception of the confused phonemes. According to other examples, the frequency allocation of each channel of the filterbank of the processor of a cochlear implant can be changed to ensure that the two confused phonemes fall into different bands, thus enhancing the contrast between the phonemes in the recipient's perception. If, on the other hand, the confused phonemes mostly differ in the temporal domain, the dynamic range of stimulation can be increased, thus increasing the sensitivity to amplitude modulation and enhancing the difference between the phonemes in the recipient's perception. Similar to the previous example, the loudness compression in the recipient's cochlear implant can be changed in general, or in a specific spectral channel, to enhance the differences between the confused phonemes.

Other test results may indicate that a rehabilitative intervention, such as an individualized rehabilitation, based on the recipient's unique error profile, is more likely to result in a better outcome for the recipient. For example, if two sounds are discriminated, but they are confused by the recipient, a rehabilitative training aimed at better identification of the two perceptually different stimuli can be prescribed. Based on this determination, the identified intervention can be prescribed to the recipient. According to specific examples of the disclosed techniques, if the recipient exhibits a consistent error, such as a non-random error, with respect to a particular speech sound, a technological intervention can be determined as the best intervention for the recipient. On the other hand, an inconsistent error, such as a random error, with respect to a particular speech sound can indicate that a rehabilitative intervention should be prescribed to the recipient. Other errors or other factors can result in reaching different conclusions. For example, if a recipient exhibits a consistent error with respect to a speech sound, other factors in the way the error presents itself can result in the prescription of a rehabilitative intervention. Similarly, if a recipient exhibits an inconsistent error with respect to a speech sound, other factors in the way the error presents itself can result in the prescription of a technological intervention.

As noted above, the disclosed techniques can be used with a number of different hearing devices and/or other types of implantable medical devices. Merely for ease of description, reference is generally made to use of the disclosed techniques with hearing prosthesis recipients. As such, reference to hearing prostheses and/or hearing prostheses recipients is merely illustrative and does not limit the scope of the invention to any particular use.

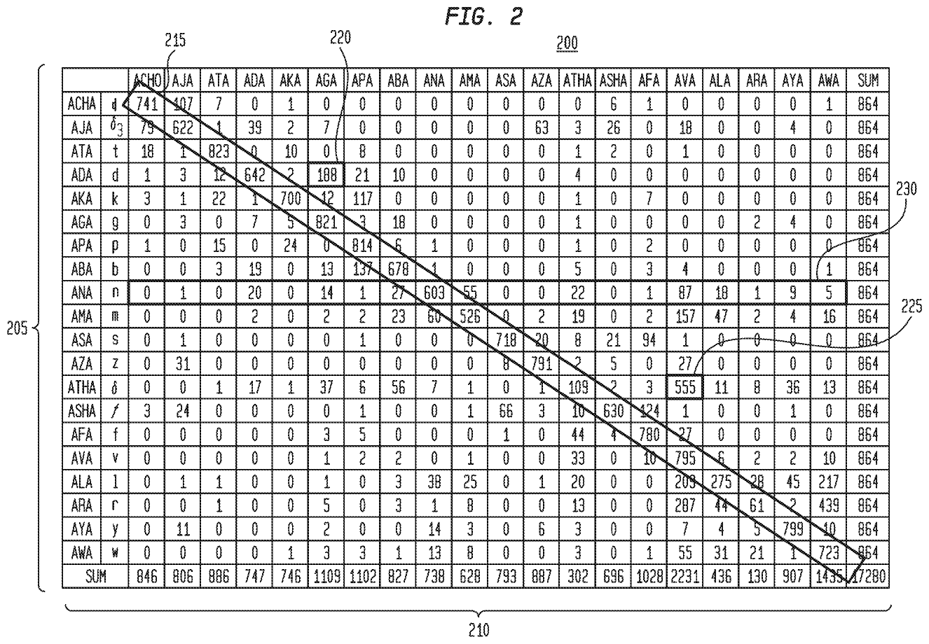

The disclosed techniques begin with presenting a hearing prosthesis recipient with a diagnostic test battery of speech sounds. According to specific examples, the battery of test sounds can include a consonant and/or vowel phoneme identification test. Consonant and vowel phoneme tests can measure the subject's ability to identify vowels and consonants in a closed-set context. For consonants, an auditory stimulus of the “vCv” type can be presented, where “v” is a vowel sound and “C” is the consonant that is being tested for. For vowels, a stimulus of the ‘hVd’ type can be presented, where “V” represents the vowel of interest and “h” and “d” represent the /h/ and /d/ phonemes respectively. In both test conditions (vowels and consonants), the recipient can be presented with a list of possible choices and will select which one they think they heard. Because the test subject's choices are limited, this type of test is referred to as a “closed set” test. The results of the test can be compiled into a phoneme confusion matrix for vowels and consonants, an example of which is illustrated in confusion matrix 200 of FIG. 2. Input (the presented stimulus) is seen on the Y-axis 205, and output (the reported stimulus) is seen on the X-axis 210. Correct answers are shown on the diagonal axis 215, errors are shown in the off-diagonal elements. For example, in diagonal axis 215, the input from the Y-axis 205 matches the recipient's output from the X-axis 210, and therefore, the numbers in the diagonal axis 215 represent the number of correct responses by the recipient. Numbers outside of diagonal axis 215 indicate which phoneme was incorrectly identified and how many times.

Traditionally, in clinical evaluation of a recipient's performance level, the recipient errors in consonant and/or vowel phoneme identification tests have only taken into account the overall quantitative results of the test, such as evaluating the overall percent of answers correct by the recipient. In contrast, the disclosed techniques evaluate specific features of the test results to determine specific error patterns for specific speech sounds. For example, applying the disclosed techniques to confusion matrix 200 allows for the identification of specific error types for specific speech sounds. According to this specific example, confusion matrix 200 can be used to identify and distinguish between non-random error patterns and random error patterns for the specific phonemes illustrated therein.

In a non-random error pattern, if an error is made against a specific input, these inputs are consistently confused with an alternative option. For example, the test results illustrated in confusion matrix 200 illustrate that an /ada/ phoneme input is consistently confused with the /aga/ phoneme. As shown through entry 220 in confusion matrix 200, the recipient confused the /ada/ phoneme input with the /afa/ phoneme 188 times. Similarly, as shown through entry 225 in confusion matrix 200, the recipient confused the /atha/ phoneme input with the /ava/ phoneme 555 times. These errors can be identified as consistent or non-random errors. In a random error pattern, if an error is made against a specific input, the outputs are non-consistent. For example, the output of the /ana/ phoneme is spread across /ada/, /aga/, /aba/, and other phonemes, as shown in row 230.

As illustrated through confusion matrix 200, the disclosed techniques can analyze the content of confusion matrix 200 to identify trends in recipient responses, and in response thereto, determine appropriate and/or individualized interventions. Through such individualized interventions, the identified errors can be overcome or compensated for through targeted device settings and/or training.

The analysis of confusion matrix 200 can be performed in numerous ways without deviating from the disclosed techniques. For example, an individual clinician can analyze confusion matrix 200 to identify the non-random errors illustrated through entries 220 and 225 and the random error illustrated through row 230. The analysis of confusion matrix 200 can also be automated using a statistical data analysis algorithm running on a processing device, which may be the same or a different processing device than the device used to administer the diagnostic test. For example, confusion matrix 200 can be analyzed as a heat map via a processing device, with the processing device being configured to identify maxima values as non-random errors and identify horizontal contour lines as random errors.

According to other example embodiments, the analysis of confusion matrix 200 can be performed such that it identifies errors that are unique or uncommon within the population of recipients of a particular hearing device, such as the population of hearing aid or cochlear implant recipients. For example, as shown through entry 225 in confusion matrix 200, the recipient confused the /atha/ phoneme input with the /ava/ phoneme 555 times. If this is a relatively common error, then no intervention or only generalized interventions may be prescribed to the recipient. However, if this is an uncommon error or an error unique to this particular recipient, then this error can be flagged during the analysis of confusion matrix 200 for further analysis, and potentially, additional intervention.

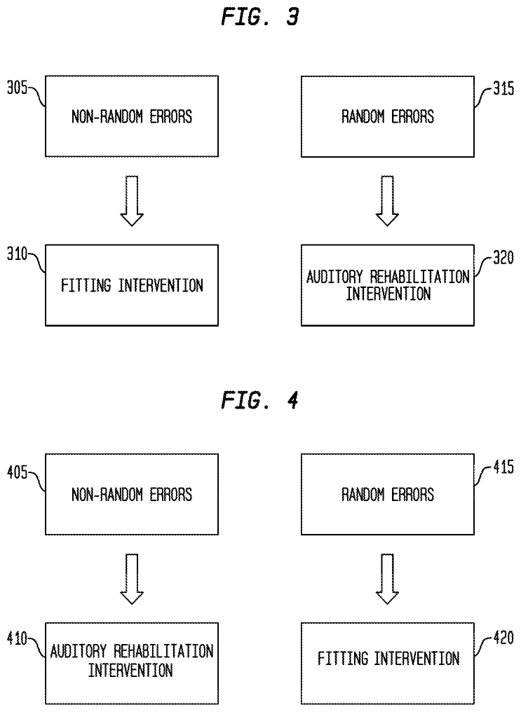

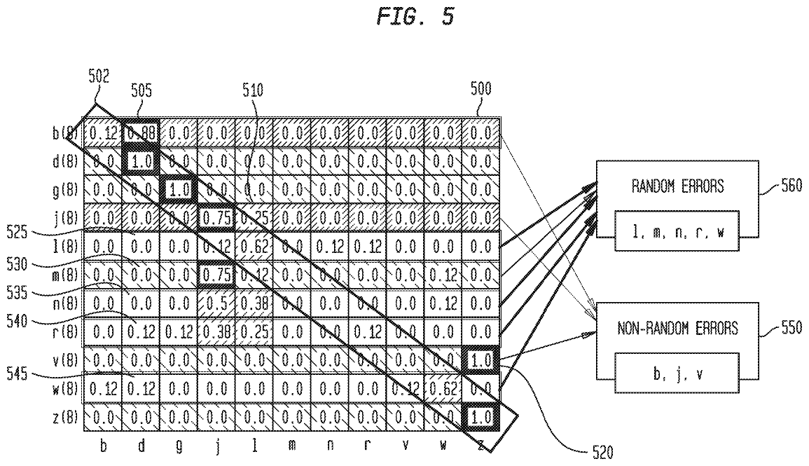

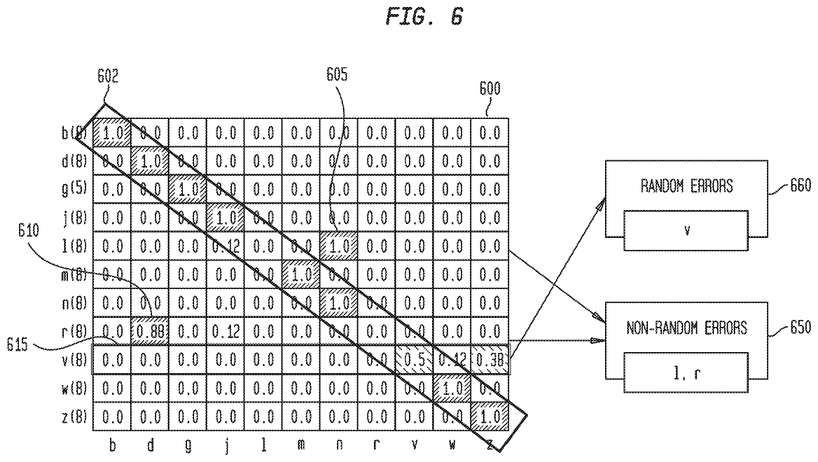

Turning now to FIG. 3, specific diagnostic test batteries can be designed such that non-random errors in the test results can be addressed through technological interventions (e.g., fitting interventions), while random errors in the test results can be addressed through rehabilitation interventions. Accordingly, as illustrated in FIG. 3, when a non-random error 305 with respect to a specific phoneme is identified a technological intervention, in this case a cochlear implant fitting intervention 310, is prescribed to the recipient. According to specific examples, non-random errors that cross frequency ranges associated with the electrodes of a cochlear implant can be addressed through fitting remediation. A random error 315 with respect to a specific phoneme in the same diagnostic test can result in the prescription of auditory rehabilitation intervention 320. FIG. 4, on the other hand, illustrates a different diagnostic test in which a non-random error 405 is prescribed a technological or fitting intervention 410, and in which a random error 415 is prescribed an auditory rehabilitation intervention 420. Accordingly, the specific type of intervention prescribed in response to an identified random or non-random error can be specific to the diagnostic test performed, the auditory stimuli presented during the test, and/or features of the recipient's auditory or prosthesis capabilities.

The disclosed techniques for designing specific interventions for random vs. non-random errors as illustrated in FIGS. 3 and 4 can also be applied to designing techniques for a recipient's uncommon or unique errors identified through analysis of a confusion matrix. For example, analysis of an uncommon error, alone or in combination with other common or uncommon errors identified for the recipient, can be used to identify and prescribe a particular intervention for the recipient. For example, if the uncommon error indicates a fitting issue of the recipient, then a fitting intervention can be prescribed to address the uncommon error. If, on the other hand, the uncommon error indicates a behavioral issue of the recipient, a rehabilitative intervention can be prescribed for the recipient.

Turning to FIG. 5, depicted therein is a confusion matrix 500 illustrating the results of a diagnostic test administered to a first recipient. According to this specific example, the recipient is provided with a computer application, such as an application executing on a mobile phone. The application can be configured to interface with the first recipient's hearing prosthesis and cause the hearing prosthesis to initiate a battery of hearing tests by presenting a series of target phonemes to the recipient. According to other examples, the mobile phone presents the target phonemes via its internal speaker without directly interfacing with the hearing prosthesis. After hearing a target phoneme, the recipient is prompted to indicate what they heard by selecting one of several visually presented options on the user interface of the mobile phone. In other words, the recipient is presented with a closed set or closed response test. Closed set/closed response tests may be applicable to the techniques disclosed herein as recipients are forced to select an answer. Recipients can omit responses in open set tests making it more difficult to distinguish between random and non-random errors. It can be beneficial to present a large number of options to the user, such as 12 options, to ensure that the non-random errors are appropriately identified. Similarly, each phoneme can be presented to the user multiple times, such as 8 times, to provide sufficient test data to distinguish between random and non-random errors. Furthermore, the test stimuli can be presented to the recipient in random order. According to other examples, the stimuli can be presented to the recipient based upon the output of a machine learning algorithm. Such an algorithm can determine “problem areas” for the recipient and select and present stimuli to the recipient to evaluate such “problem areas” during the diagnostic test. The use of such an algorithm can decrease the length of diagnostic tests, improving recipient satisfaction with their care.

Upon completion of the diagnostic test, confusion matrix 500 is generated. Confusion matrix 500 differs from confusion matrix 200 of FIG. 2 in that the values in confusion matrix 500 have been normalized to 1, while the values in confusion matrix 200 are the unnormalized number of errors. Once the hearing tests are completed, the results are analyzed to identify any specific phonemes that are consistently misheard in a non-random manner. Based upon the values contained in confusion matrix 500, entries 505, 510, and 520, associated with consonant phonemes “b,” “j” and “v” respectively, are identified as non-random errors as they indicate substantial answers by the recipient outside of diagonal axis 502 with a consistent incorrect response. Accordingly, these values are mapped as non-random errors 550. For example, the recipient is consistently mishearing the test phoneme “b” as “d” as illustrated in entry 505. Insofar as such non-random errors are identified, the application can initiate a re-fitting module which permits the recipient or a clinician to make device adjustments to the recipient's hearing prosthesis with specific attention to correcting the misheard phoneme.

The hearing test results can also be analyzed to identify any test phonemes that are misheard in a random manner. Rows 525, 530, 535, 540 and 545, associated with consonant phonemes “l,” “m,” “n,” “r” and “w” respectively, are identified as random errors 560 as they indicate substantial answers outside of diagonal axis 502 with inconsistent incorrect responses, i.e., substantial incorrect responses in more than one of the columns outside of the diagonal axis 502. For example, as illustrated in row 525, the recipient is consistently mishearing the test phoneme “1”, but has responded inconsistently by selecting the “j,” “n” or “r” phonemes at different times. Insofar as such random errors are identified, the application can initiate a rehabilitation module which provides the recipient with targeted auditory practice exercises with a goal of helping the recipient to more consistently hear the “l” phoneme correctly.

With reference now made to FIG. 6, depicted therein is a confusion matrix 600 that exhibits both random and non-random errors, but to a lesser extent than confusion matrix 500 of FIG. 5. Accordingly, a traditional analysis of confusion matrix 600, in which only the total number of incorrect responses is analyzed, could result in the recipient not receiving appropriately tailored or individualized interventions. For example, because there are significantly fewer errors in confusion matrix 600 than confusion matrix 500, the specific errors associated with the recipient can be overlooked utilizing traditional confusion matrix analysis techniques.

However, by utilizing the techniques disclosed herein, it can be determined that the recipient whose answers populate confusion matrix 600 requires technological interventions to address errors associated with the “l” and “r” phonemes and rehabilitative interventions with respect to the “v” phoneme. As illustrated in confusion matrix 600, entries 605 and 610, associated with the “l” and “r” consonant phonemes, respectively, are identified as non-random errors as they indicate substantial answers by the recipient outside of diagonal axis 602 with a consistent incorrect response. Accordingly, these values are mapped as non-random errors 650. In response to the identification of these non-random errors, the application administering the diagnostic test can initiate a re-fitting module which permits the recipient or a clinician to make device adjustments with specific attention to correcting perception and identification of the “l” and “r” consonant phonemes.

Utilizing the techniques disclosed herein can also provide the recipient with rehabilitative interventions that may have been overlooked using traditional confusion matrix techniques. Specifically, row 615 associated with the “v” consonant phoneme is identified as a random error 660 because row 615 indicates substantial answers outside of diagonal axis 602 with inconsistent incorrect responses, i.e., substantial incorrect responses in more than one of the columns outside of the diagonal axis 602. The application can initiate a rehabilitation module which provides the recipient with targeted auditory practice exercises with a goal of helping the recipient to more consistently hear and identify the “v” phoneme correctly.

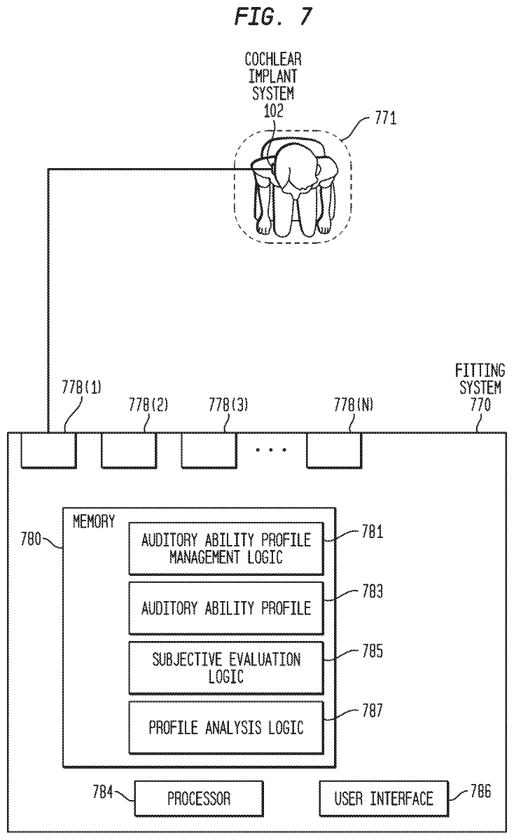

As indicated above, the technological interventions described herein can include a cochlear implant fitting intervention. Accordingly, illustrated in FIG. 7 is a block diagram illustrating an example fitting system 770 configured to execute the techniques presented herein. Fitting system 770 is, in general, a computing device that comprises a plurality of interfaces/ports 778(1)-778(N), a memory 780, a processor 784, and a user interface 786. The interfaces 778(1)-778(N) can comprise, for example, any combination of network ports (e.g., Ethernet ports), wireless network interfaces, Universal Serial Bus (USB) ports, Institute of Electrical and Electronics Engineers (IEEE) 1394 interfaces, PS/2 ports, etc. In the example of FIG. 7, interface 778(1) is connected to cochlear implant system 102 having components implanted in a recipient 771. Interface 778(1) can be directly connected to the cochlear implant system 102 or connected to an external device that is communicating with the cochlear implant systems. Interface 778(1) can be configured to communicate with cochlear implant system 102 via a wired or wireless connection (e.g., telemetry, Bluetooth, etc.).

The user interface 786 includes one or more output devices, such as a display screen (e.g., a liquid crystal display (LCD)) and a speaker, for presentation of visual or audible information to a clinician, audiologist, or other user. The user interface 786 can also comprise one or more input devices that include, for example, a keypad, keyboard, mouse, touchscreen, etc.

The memory 780 comprises auditory ability profile management logic 781 that can be executed to generate or update a recipient's auditory ability profile 783 that is stored in the memory 780. The auditory ability profile management logic 781 can be executed to obtain the results of objective evaluations of a recipient's cognitive auditory ability from an external device, such as an imaging system (not shown in FIG. 7), via one of the other interfaces 778(2)-778(N). In certain embodiments, memory 780 comprises subjective evaluation logic 785 that is configured to perform subjective evaluations of a recipient's cognitive auditory ability and provide the results for use by the auditory ability profile management logic 781. Accordingly, auditory ability profile management logic 781 can include logic configured to execute and analyze a diagnostic test in accordance with the techniques disclosed herein. In other embodiments, the subjective evaluation logic 785 is omitted and the auditory ability profile management logic 781 is executed to obtain the results of subjective evaluations of a recipient's cognitive auditory ability from an external device (not shown in FIG. 7), via one of the other interfaces 778(2)-778(N). Similarly, a diagnostic test in accordance with the techniques disclosed herein can be executed and analyzed from the external device.

The memory 780 further comprises profile analysis logic 787. The profile analysis logic 787 is executed to analyze the recipient's auditory profile (i.e., the correlated results of the objective and subjective evaluations) to identify correlated stimulation parameters that are optimized for the recipient's cognitive auditory ability. Profile analysis logic 787 can also be configured to identify stimulation parameters based upon the analysis of a diagnostic test in accordance with the techniques disclosed herein.

Memory 780 can comprise read only memory (ROM), random access memory (RAM), magnetic disk storage media devices, optical storage media devices, flash memory devices, electrical, optical, or other physical/tangible memory storage devices. The processor 784 is, for example, a microprocessor or microcontroller that executes instructions for the auditory ability profile management logic 781, the subjective evaluation logic 785, and the profile analysis logic 787. Thus, in general, the memory 780 can comprise one or more tangible (non-transitory) computer readable storage media (e.g., a memory device) encoded with software comprising computer executable instructions and when the software is executed (by the processor 784) it is operable to perform the techniques described herein.

The correlated stimulation parameters identified through execution of the profile analysis logic 787 are sent to the cochlear implant system 102 for instantiation as the cochlear implant's current correlated stimulation parameters. Accordingly, fitting system 770 can implement a cochlear implant fitting intervention determined according to the techniques disclosed herein. However, in certain embodiments, the correlated stimulation parameters identified through execution of the profile analysis logic 787 are first displayed at the user interface 786 for further evaluation and/or adjustment by a user. As such, the user (e.g., an audiologist or the cochlear implant recipient) has the ability to refine the correlated stimulation parameters before the stimulation parameters are sent to the cochlear implant system 102.

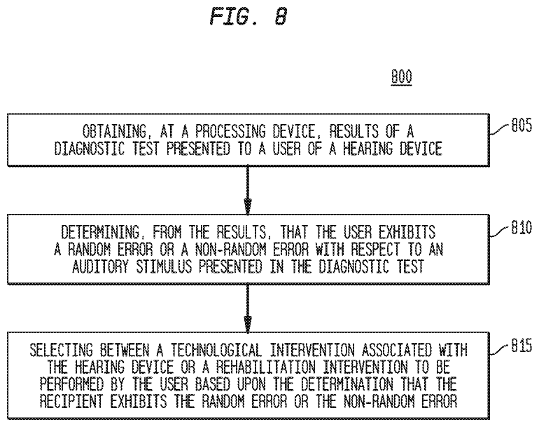

With reference now made to FIG. 8, depicted therein is a flowchart 800 illustrating a generalized process flow for implementing the phoneme error based intervention techniques of this disclosure. Flowchart 800 begins in operation 805 in which the results of a diagnostic test are obtained. The diagnostic test was presented to a recipient of a hearing prosthesis. According to specific examples, the diagnostic test may have been based upon a diagnostic test battery that presented speech sound auditory stimuli to the recipient. The speech sound auditory stimuli may have included phonemes or tonemes of a tonal language. The diagnostic test presented to the recipient may have been a closed response diagnostic test in which the recipient was presented with a plurality of possible response and was required to select the response corresponding to the speech sound auditory stimuli presented to them. The results may have been compiled into a speech confusion matrix. In other words, the results obtained in operation 805 may be embodied as the results of one or more of the different types of diagnostic tests described above with reference to FIGS. 2-6.

In operation 810, it is determined from the results that the recipient exhibits a random error or a non-random error with respect to an auditory stimulus present in the diagnostic test. Accordingly, operation 810 can be embodied as the identification of an error as described above with reference to FIGS. 2-6. For example, operation 810 can be embodied as identifying a random error, a non-random error, or both in a confusion matrix as described above with reference to FIGS. 2, 5 and 6.

Finally, in operation 815, a selection is made between a technological intervention associated with the hearing prosthesis or a rehabilitation intervention to be performed by the recipient. The selection is based upon the determination that the recipient exhibits the random error or the non-random error. For example, the selection of operation 815 can select a technological intervention in response to determining a non-random error in operation 810, as illustrated in FIG. 3 above. Alternatively, the selection of operation 815 can select a technological intervention in response to determining a random error in operation 810, as illustrated in FIG. 4 above. Similarly, operation 815 can select a rehabilitative intervention in response to determining a random error in operation 810, as illustrated in FIG. 3 above. Alternatively, the selection of operation 815 can select a rehabilitative intervention in response to determining a non-random error in operation 810, as illustrated in FIG. 4 above.

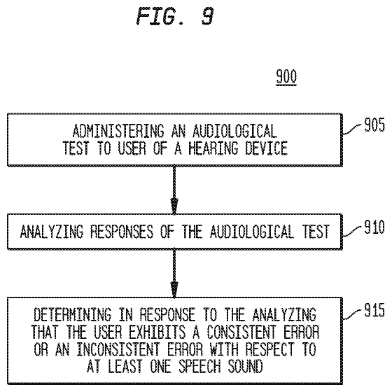

As shown through the discussion above, flowchart 800 can implement the error identification and intervention selection aspects of the disclosed techniques. Flowchart 900 of FIG. 9, on the other hand, illustrates a process flow for implementing the diagnostic test administration and analysis aspects of the disclosed techniques.

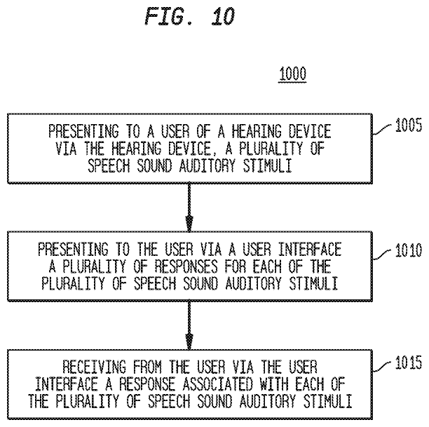

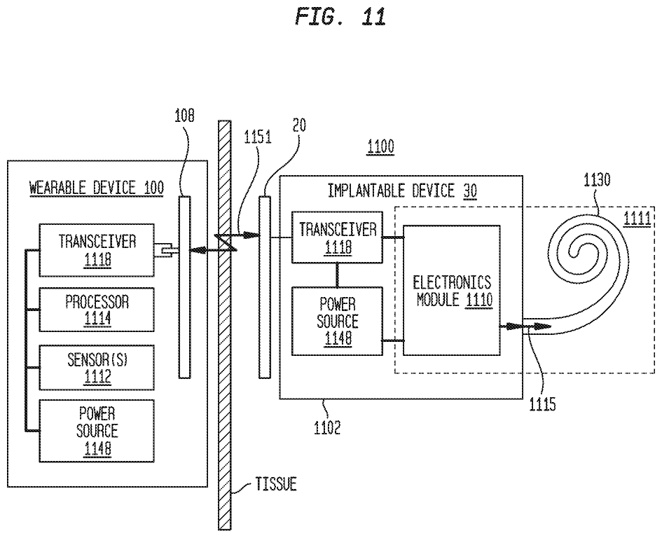

Flowchart 900 begins in operation 905 in which an audiological test is administered to a recipient of a hearing prosthesis. Accordingly, operation 905 can be embodied as the administration of a diagnostic battery as described above with reference to FIGS. 2-6. Turning briefly to FIG. 10, depicted therein is a flowchart 1000 illustrating a process flow for implementing a specific example of administering an audiological test. Flowchart 1000 begins in operation 1005 in which a plurality of speech sound auditory stimuli are presented to a recipient of a hearing prosthesis. The auditory stimuli are presented to the recipient by the hearing prosthesis. Operation 1005 should be interpreted broadly such that the presentation of the auditory stimuli of operation 1005 can encompass presenting auditory stimuli generated by the hearing prosthesis itself or the presentation of auditory stimuli generated by another device and transmitted to the recipient by the hearing prosthesis.

In operation 1010, the recipient is presented with a plurality of responses for each of the plurality of speech sound auditory stimuli via a user interface. For example, operation 1010 can be embodied as the presentation of responses as part of a closed response diagnostic test. The user interface of operation 1010 can be the screen of a personal computing device, including the touchscreen of a smartphone or tablet computing device. When implemented through such a personal computing device, recipients have the opportunity to conduct the audiological test at their own pace. The user interface of operation 1010 can also be the user interface of a fitting system as described above with reference to FIG. 7. Finally, in operation 1015, a response for each of the plurality of speech sound auditory stimuli is received from the recipient via the user interface.

Returning to FIG. 9, the process flow of flowchart 900 proceeds from operation 905 (whether implemented via flowchart 1000 of FIG. 10 or via another process) to operation 910. In operation 910, responses of the audiological test are analyzed. Accordingly, operation 910 can be embodied as the compiling of the responses into a speech confusion matrix as described above with reference to FIGS. 2, 5 and 6.

Finally, in operation 915, it is determined in response to the analysis of operation 910 that the recipient exhibits a consistent error or an inconsistent error with respect to at least one speech sound. As described above, the speech sound can be a phoneme, a toneme, a consonant-vowel-consonant speech sound, a vowel-consonant-vowel speech sound, a spondee, a word, or combinations thereof.

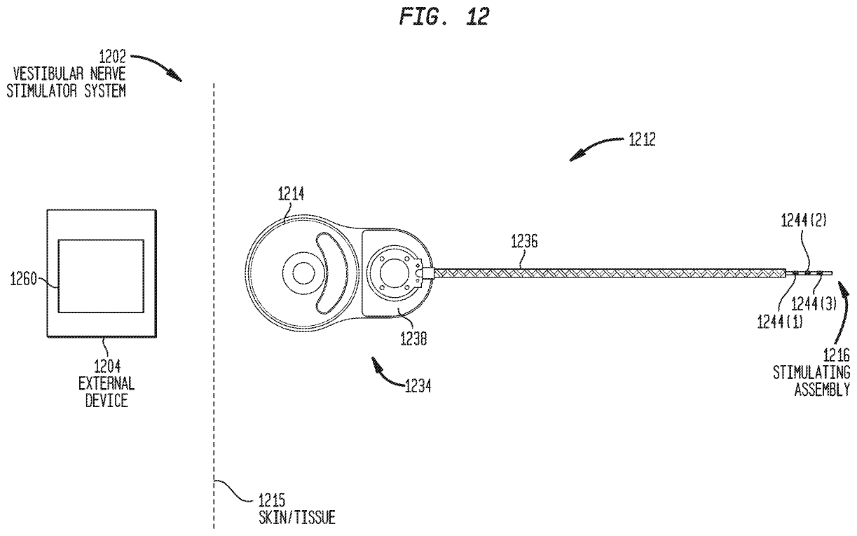

As previously described, the technology disclosed herein can be applied in any of a variety of circumstances and with a variety of different devices. Example devices that can benefit from technology disclosed herein are described in more detail in FIGS. 11 and 12, below. As described below, the operating parameters for the devices described with reference to FIGS. 11 and 12 can be configured using a fitting system analogous to fitting system 770 of FIG. 7. For example, the techniques described herein can be used to prioritize clinician tasks associated with configuring the operating parameters of wearable medical devices, such as an implantable stimulation system as described in FIG. 11 or a vestibular stimulator as described in FIG. 12. The techniques of the present disclosure can be applied to other medical devices, such as neurostimulators and vestibular stimulation devices, as well as other medical devices that deliver stimulation to tissue. Further, technology described herein can also be applied to consumer devices. These different systems and devices can benefit from the technology described herein.

FIG. 11 is a functional block diagram of an implantable stimulator system 1100 that can benefit from the technologies described herein. The implantable stimulator system 1100 includes the wearable device 100 acting as an external processor device and an implantable device 30 acting as an implanted stimulator device. In examples, the implantable device 30 is an implantable stimulator device configured to be implanted beneath a recipient's tissue (e.g., skin). In examples, the implantable device 30 includes a biocompatible implantable housing 1102. Here, the wearable device 100 is configured to transcutaneously couple with the implantable device 30 via a wireless connection to provide additional functionality to the implantable device 30.

In the illustrated example, the wearable device 100 includes one or more sensors 1112, a processor 1114, a transceiver 1118, and a power source 1148. The one or more sensors 1112 can be one or more units configured to produce data based on sensed activities. In an example where the stimulation system 1100 is an auditory prosthesis system, the one or more sensors 1112 include sound input sensors, such as a microphone, an electrical input for an FM hearing system, other components for receiving sound input, or combinations thereof. Where the stimulation system 1100 is a visual prosthesis system, the one or more sensors 1112 can include one or more cameras or other visual sensors. Where the stimulation system 1100 is a cardiac stimulator, the one or more sensors 1112 can include cardiac monitors. The processor 1114 can be a component (e.g., a central processing unit) configured to control stimulation provided by the implantable device 30. The stimulation can be controlled based on data from the sensor 1112, a stimulation schedule, or other data. Where the stimulation system 1100 is an auditory prosthesis, the processor 1114 can be configured to convert sound signals received from the sensor(s) 1112 (e.g., acting as a sound input unit) into signals 1151. The transceiver 1118 is configured to send the signals 1151 in the form of power signals, data signals, combinations thereof (e.g., by interleaving the signals), or other signals. The transceiver 1118 can also be configured to receive power or data. Stimulation signals can be generated by the processor 1114 and transmitted, using the transceiver 1118, to the implantable device 30 for use in providing stimulation.

In the illustrated example, the implantable device 30 includes a transceiver 1118, a power source 1148, and a medical instrument 1111 that includes an electronics module 1110 and a stimulator assembly 1130. The implantable device 30 further includes a hermetically sealed, biocompatible implantable housing 1102 enclosing one or more of the components.

The electronics module 1110 can include one or more other components to provide medical device functionality. In many examples, the electronics module 1110 includes one or more components for receiving a signal and converting the signal into the stimulation signal 1115. The electronics module 1110 can further include a stimulator unit. The electronics module 1110 can generate or control delivery of the stimulation signals 1115 to the stimulator assembly 1130. In examples, the electronics module 1110 includes one or more processors (e.g., central processing units or microcontrollers) coupled to memory components (e.g., flash memory) storing instructions that when executed cause performance of an operation. In examples, the electronics module 1110 generates and monitors parameters associated with generating and delivering the stimulus (e.g., output voltage, output current, or line impedance). In examples, the electronics module 1110 generates a telemetry signal (e.g., a data signal) that includes telemetry data. The electronics module 1110 can send the telemetry signal to the wearable device 100 or store the telemetry signal in memory for later use or retrieval.

The stimulator assembly 1130 can be a component configured to provide stimulation to target tissue. In the illustrated example, the stimulator assembly 1130 is an electrode assembly that includes an array of electrode contacts disposed on a lead. The lead can be disposed proximate tissue to be stimulated. Where the system 1100 is a cochlear implant system, the stimulator assembly 1130 can be inserted into the recipient's cochlea. The stimulator assembly 1130 can be configured to deliver stimulation signals 1115 (e.g., electrical stimulation signals) generated by the electronics module 1110 to the cochlea to cause the recipient to experience a hearing percept. In other examples, the stimulator assembly 1130 is a vibratory actuator disposed inside or outside of a housing of the implantable device 30 and configured to generate vibrations. The vibratory actuator receives the stimulation signals 1115 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 1118 can be components configured to transcutaneously receive and/or transmit a signal 1151 (e.g., a power signal and/or a data signal). The transceiver 1118 can be a collection of one or more components that form part of a transcutaneous energy or data transfer system to transfer the signal 1151 between the wearable device 100 and the implantable device 30. Various types of signal transfer, such as electromagnetic, capacitive, and inductive transfer, can be used to usably receive or transmit the signal 1151. The transceiver 1118 can include or be electrically connected to a coil 20.

As illustrated, the wearable device 100 includes a coil 108 for transcutaneous transfer of signals with the concave coil 20. As noted above, the transcutaneous transfer of signals between coil 108 and the coil 20 can include the transfer of power and/or data from the coil 108 to the coil 20 and/or the transfer of data from coil 20 to the coil 108. The power source 1148 can be one or more components configured to provide operational power to other components. The power source 1148 can be or include one or more rechargeable batteries. Power for the batteries can be received from a source and stored in the battery. The power can then be distributed to the other components as needed for operation.

As should be appreciated, while particular components are described in conjunction with FIG. 11, technology disclosed herein can be applied in any of a variety of circumstances. The above discussion is not meant to suggest that the disclosed techniques are only suitable for implementation within systems akin to that illustrated in and described with respect to FIG. 11. In general, additional configurations can be used to practice the methods and systems herein and/or some aspects described can be excluded without departing from the methods and systems disclosed herein.

FIG. 12 illustrates an example vestibular stimulator system 1202, with which embodiments presented herein can be implemented. As shown, the vestibular stimulator system 1202 comprises an implantable component (vestibular stimulator) 1212 and an external device/component 1204 (e.g., external processing device, battery charger, remote control, etc.). The external device 1204 comprises a transceiver unit 1260. As such, the external device 1204 is configured to transfer data (and potentially power) to the vestibular stimulator 1212, The vestibular stimulator 1212 comprises an implant body (main module) 1234, a lead region 1236, and a stimulating assembly 1216, all configured to be implanted under the skin/tissue (tissue) 1215 of the recipient. The implant body 1234 generally comprises a hermetically-sealed housing 1238 in which RF interface circuitry, one or more rechargeable batteries, one or more processors, and a stimulator unit are disposed. The implant body 134 also includes an internal/implantable coil 1214 that is generally external to the housing 1238, but which is connected to the transceiver via a hermetic feedthrough (not shown).

The stimulating assembly 1216 comprises a plurality of electrodes 1244(1)-(3) disposed in a carrier member (e.g., a flexible silicone body). In this specific example, the stimulating assembly 1216 comprises three (3) stimulation electrodes, referred to as stimulation electrodes 1244(1), 1244(2), and 1244(3). The stimulation electrodes 1244(1), 1244(2), and 1244(3) function as an electrical interface for delivery of electrical stimulation signals to the recipient's vestibular system.

The stimulating assembly 1216 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 1212, the external device 1204, and/or another external device, can be configured to implement the techniques presented herein. That is, the vestibular stimulator 1212, possibly in combination with the external device 1204 and/or another external device, can include an evoked biological response analysis system, as described elsewhere herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

In summary, by having cochlear implant recipients first undergo a diagnostic test to determine their user-specific phoneme perception errors and error patterns, individualized intervention to target these errors and error patterns can be implemented. Based on these errors and error patterns, individualized aftercare is developed. This targeted, individualized aftercare aims to improve outcomes of cochlear implant recipient, and adult recipients in particular. These techniques can be particularly beneficial to cochlear implant recipients in “poor performer” groups. By implementing the disclosed techniques, long-term objectives of rehabilitation can be facilitated, including integrating recipients back into society, providing recipients with skills and training that allows for equal opportunities compared to normal hearing individuals, and improving recipients'overall quality of life.

Accordingly, in some aspects, the techniques described herein relate to a method, including: obtaining, at a processing device, results of a diagnostic test presented to a recipient of a hearing prosthesis; determining, from the results, that the recipient exhibits a random error or a non-random error with respect to an auditory stimulus presented in the diagnostic test; and selecting between a technological intervention associated with the hearing prosthesis or a rehabilitation intervention to be performed by the recipient based upon the determination that the recipient exhibits the random error or the non-random error.

In some aspects, the techniques described herein relate to a method, wherein the selecting includes selecting the technological intervention in response to determining that the recipient exhibits the random error.

In some aspects, the techniques described herein relate to a method: wherein the hearing prosthesis includes a cochlear implant; wherein the non-random error includes non-random errors that cross frequency ranges associated with electrodes of the hearing prosthesis; and wherein the technological intervention includes a fitting of the cochlear implant.

In some aspects, the techniques described herein relate to a method, wherein the selecting includes selecting the rehabilitation intervention in response to determining that the recipient exhibits the non-random error.

In some aspects, the techniques described herein relate to a method, wherein the auditory stimulus includes a phoneme.

In some aspects, the techniques described herein relate to a method, wherein the auditory stimulus includes a toneme.

In some aspects, the techniques described herein relate to a method, wherein the diagnostic test includes a closed response diagnostic test.

In some aspects, the techniques described herein relate to a method, wherein the diagnostic test includes an audiological test.

In some aspects, the techniques described herein relate to a method, wherein the audiological test includes a speech test.

In some aspects, the techniques described herein relate to a method including:
administering an audiological test to a recipient of a hearing prosthesis, the administering including: presenting to the recipient via the hearing prosthesis, a plurality of speech sound auditory stimuli, presenting to the recipient via a user interface a plurality of responses for each of the plurality of speech sound auditory stimuli, and receiving from the recipient via the user interface, a response associated with each of the plurality of speech sound auditory stimuli;
analyzing the responses associated with each of the plurality of speech sound auditory stimuli;
and determining in response to the analyzing that the recipient exhibits a consistent error or an inconsistent error with respect to at least one of the plurality of speech sound auditory stimuli.

In some aspects, the techniques described herein relate to a method, further including selecting a technological intervention in response determining that the recipient exhibits the consistent error with respect to the at least one of the plurality of speech sound auditory stimuli.

In some aspects, the techniques described herein relate to a method, wherein the consistent error includes a non-random error.

In some aspects, the techniques described herein relate to a method, wherein the technological intervention includes a cochlear implant fitting intervention.

In some aspects, the techniques described herein relate to a method, further including selecting a rehabilitation intervention in response to the determining that the recipient exhibits the inconsistent error with respect to the at least one of the plurality of speech sound auditory stimuli.

In some aspects, the techniques described herein relate to a method, wherein the inconsistent error includes a non-random error.

In some aspects, the techniques described herein relate to a method, wherein the plurality of speech sound auditory stimuli include a plurality of phoneme stimuli.

In some aspects, the techniques described herein relate to a method, wherein the plurality of speech sound auditory stimuli include a plurality of toneme stimuli.

In some aspects, the techniques described herein relate to a method, wherein the user interface includes a personal computing device.

In some aspects, the techniques described herein relate to a method, wherein the personal computing device includes a smartphone or tablet computing device.

In some aspects, the techniques described herein relate to a method, wherein the personal computing device is configured to interface with the hearing prosthesis to induce the hearing prosthesis to deliver the plurality of speech sound auditory stimuli to the recipient via the hearing prosthesis.

In some aspects, the techniques described herein relate to one or more non-transitory computer readable storage media including instructions that, when executed by a processor, cause the processor to: obtain results of a diagnostic test presented to a recipient of a hearing prosthesis; determine, from the results, that the recipient exhibits a random error or a non-random error with respect to an auditory stimulus presented in the diagnostic test; and select between a technological intervention associated with the hearing prosthesis or a rehabilitation intervention to be performed by the recipient based upon the determination that the recipient exhibits the random error or the non-random error.

In some aspects, the techniques described herein relate to one or more non-transitory computer readable storage media, wherein the instructions that cause the processor to select the technological intervention associated with the hearing prosthesis or the rehabilitation intervention to be performed by the recipient include instructions that cause the processor to select the technological intervention in response to determining that the recipient exhibits the random error.

In some aspects, the techniques described herein relate to one or more non-transitory computer readable storage media: wherein the hearing prosthesis includes a cochlear implant; wherein the non-random error includes non-random errors that cross frequency ranges associated with electrodes of the hearing prosthesis; and wherein the technological intervention includes a fitting of the cochlear implant.

In some aspects, the techniques described herein relate to one or more non-transitory computer readable storage media, wherein the instructions that cause the processor to select the technological intervention associated with the hearing prosthesis or the rehabilitation intervention to be performed by the recipient include instructions that cause the processor to select the rehabilitation intervention in response to determining that the recipient exhibits the non-random error.

In some aspects, the techniques described herein relate to one or more non-transitory computer readable storage media, wherein the auditory stimulus includes a phoneme.

In some aspects, the techniques described herein relate to one or more non-transitory computer readable storage media, wherein the auditory stimulus includes a toneme.

In some aspects, the techniques described herein relate to one or more non-transitory computer readable storage media, wherein the diagnostic test includes a closed response diagnostic test.

In some aspects, the techniques described herein relate to one or more non-transitory computer readable storage media, wherein the diagnostic test includes an audiological test.

In some aspects, the techniques described herein relate to one or more non-transitory computer readable storage media, wherein the audiological test includes a speech test.

In some aspects, the techniques described herein relate to a system, including: a hearing prosthesis; and a processing device including a user interface and at least one processor, wherein the at least one processor is configured to: cause the hearing prosthesis to present a plurality of speech sound auditory stimuli to a recipient of the hearing prosthesis; present to the recipient via the user interface a plurality of responses for each of the plurality of speech sound auditory stimuli; receive from the recipient via the user interface a response associated with each of the plurality of speech sound auditory stimuli; analyze the responses associated with each of the plurality of speech sound auditory stimuli; and determine in response to the analyzing that the recipient exhibits a consistent error or an inconsistent error with respect to at least one of the plurality of speech sound auditory stimuli.

In some aspects, the techniques described herein relate to a system, wherein the at least one processor is further configured to select a technological intervention in response determining that the recipient exhibits the consistent error with respect to the at least one of the plurality of speech sound auditory stimuli.

In some aspects, the techniques described herein relate to a system, wherein the consistent error includes a non-random error.

In some aspects, the techniques described herein relate to a system, wherein the technological intervention includes a cochlear implant fitting intervention.

In some aspects, the techniques described herein relate to a system, wherein the at least one processor is further configured to select a rehabilitation intervention in response to the determining that the recipient exhibits the inconsistent error with respect to the at least one of the plurality of speech sound auditory stimuli.

In some aspects, the techniques described herein relate to a system, wherein the inconsistent error includes a non-random error.

In some aspects, the techniques described herein relate to a system, wherein the plurality of speech sound auditory stimuli include a plurality of phoneme stimuli.

In some aspects, the techniques described herein relate to a system, wherein the plurality of speech sound auditory stimuli include a plurality of toneme stimuli.

In some aspects, the techniques described herein relate to a system, wherein the user interface includes a touchscreen.

In some aspects, the techniques described herein relate to a system, wherein the processing device includes a smartphone or tablet computing device.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method, comprising:
obtaining, at a processing device, results of a diagnostic test presented to a user of a device;
determining, from the results, that the user exhibits a random error or a non-random error with respect to a stimulus presented in the diagnostic test; and
selecting between a technological intervention associated with the device or a rehabilitation intervention to be performed by the user based upon the determination that the user exhibits the random error or the non-random error.

2. The method of claim 1, wherein the selecting comprises selecting the technological intervention in response to determining that the user exhibits the random error.

3. The method of claim 2:
wherein the device comprises a hearing device;
wherein the non-random error comprises non-random errors that cross frequency ranges associated with electrodes of the hearing device; and
wherein the technological intervention comprises a fitting of the hearing device.

4. The method of claim 1, wherein the selecting comprises selecting the rehabilitation intervention in response to determining that the user exhibits the non-random error.

5. The method of claim 1, wherein the stimulus comprises at least one of a phoneme or a toneme.

6. The method of claim 1, further comprising:
adjusting at least one operation of the device based on the technological intervention associated with the device or the rehabilitation intervention to be performed by the user.

7. The method of claim 1, wherein the diagnostic test comprises at least one of a closed response diagnostic test or an audiological test.

8-20. (canceled)

21. One or more non-transitory computer readable storage media comprising instructions that, when executed by a processor, cause the processor to:
obtain results of a diagnostic test presented to a user of a device;
determine, from the results, that the user exhibits a random error or a non-random error with respect to stimulus presented in the diagnostic test; and
select between a technological intervention associated with the device or a rehabilitation intervention to be performed by the user based upon the determination that the user exhibits the random error or the non-random error.

22. The one or more non-transitory computer readable storage media of claim 21, wherein the instructions that cause the processor to select the technological intervention associated with the device or the rehabilitation intervention to be performed by the user comprise instructions that cause the processor to select the technological intervention in response to determining that the user exhibits the random error.

23. The one or more non-transitory computer readable storage media of claim 21:
wherein the device comprises a hearing device;
wherein the non-random error comprises non-random errors that cross frequency ranges associated with electrodes of the hearing device; and
wherein the technological intervention comprises a fitting of the hearing device.

24. The one or more non-transitory computer readable storage media of claim 21, wherein the instructions that cause the processor to select the technological intervention associated with the device or the rehabilitation intervention to be performed by the user comprise instructions that cause the processor to select the rehabilitation intervention in response to determining that the user exhibits the non-random error.

25. The one or more non-transitory computer readable storage media of claim 21, wherein the stimulus comprises at least one of a phoneme or a toneme.

26. The one or more non-transitory computer readable storage media of claim 21, Further comprising instructions operable to initiate an adjustment to at least one operation of the device based on the technological intervention associated with the device or the rehabilitation intervention to be performed by the user.

27. The one or more non-transitory computer readable storage media of claim 21, wherein the diagnostic test comprises at least one of a closed response diagnostic test or an audiological test.

28. (canceled)

29. (canceled)

30. A system, comprising:
a processing device comprising a user interface and at least one processor,
wherein the at least one processor is configured to:
cause a device to present a plurality of speech sound auditory stimuli to a user of the hearing device;
present to the user via the user interface a plurality of responses for each of the plurality of speech sound auditory stimuli;
receive from the user via the user interface a response associated with each of the plurality of speech sound auditory stimuli;
analyze the responses associated with each of the plurality of speech sound auditory stimuli; and
determine in response to the analyzing that the user exhibits a consistent error or an inconsistent error with respect to at least one of the plurality of speech sound auditory stimuli.

31. The system of claim 30, wherein the at least one processor is further configured to select a technological intervention in response determining that the user exhibits the consistent error with respect to the at least one of the plurality of speech sound auditory stimuli.

32. The system of claim 31, wherein the consistent error comprises a non-random error.

33. The system of claim 31, wherein the technological intervention comprises a fitting intervention.

34. The system of claim 30, wherein the at least one processor is further configured to select a rehabilitation intervention in response to the determining that the user exhibits the inconsistent error with respect to the at least one of the plurality of speech sound auditory stimuli.

35. The system of claim 34, wherein the inconsistent error comprises a non-random error.

36-40. (canceled)
